(19) United States
(12) Patent Application Publication
(10) Pub. No.: US 2011/0207744 A1
(43) Pub. Date: Aug. 25, 2011

(54) Method for treating cognitive deficits

(75) Inventors: Christina Kurre Olsen, Smorum (DK); Rene Holm, Jyllinge (DK); Christine Kau, Tastrup (DK); Birgitte Willumsen, Bronshoj (DK); Klaus Peter Hertel, Jystrup (DK); Lone Bruun, Solrod Strand (DK); Karina Krojer Soby, Jystrup (DK)

(73) Assignee: H. Lundbeck A/S, Valby-Copenhagen (DK)

(21) Appl. No.: 13/101,176

(22) Filed: May 5, 2011

Related U.S. Application Data
(62) Division of application No. 12/437,363, filed on May 7, 2009, now abandoned.
(60) Provisional application No. 61/176,392, filed on May 7, 2009.
(60) Provisional application No. 61/111,701, filed on Nov. 5, 2008.
(60) Provisional application No. 61/102,377, filed on Oct. 3, 2008.

(30) Foreign Application Priority Data
May 7, 2008 (DK) PA200800647
Oct. 3, 2008 (DK) PA200801392
Nov. 4, 2008 (DK) PA200801519

(51) Int. Cl. A61K 31/4965; A61P 25/18; A61P 25/16

(52) U.S. Cl. 514/255.03

(57) ABSTRACT

The invention relates to methods of treating cognitive dysfunction and improving cognitive functioning comprising the administration of trans-4-((1R,3S)-6-chloro-3-phenylindan-1-yl)-1,2,2-trimethylpiperazine or a pharmaceutically acceptable salt thereof to a patient in need thereof. Moreover the invention relates to an improved binder in a composition comprising 4-((1R,3S)-6-chloro-3-phenylindan-1-yl)-1,2,2-trimethylpiperazine.

### CROSS REFERENCE TO RELATED APPLICATIONS

The present application is a divisional application, which claims priority under 35 USC §120 to U.S. nonprovisional application Ser. No. 12/437,363, filed May 7, 2009, which claims priority under 35 USC §119 to provisional application Ser. No. 61/176,392, filed May 7, 2009, provisional application Ser. No. 61/111,701, filed Nov. 5, 2008, and provisional application Ser. No. 61/102,377, filed Oct. 3, 2008, and Danish application No. PA200801519, filed Nov. 4, 2008, Danish application No. PA200801392, filed Oct. 3, 2008 and Danish application No. PA200800647, filed May 7, 2008, respectively, the entire disclosure of which are incorporated herein in their entirety.

The present invention relates to the use of trans-4-((1R,3S)-6-chloro-3-phenylindan-1-yl)-1,2,2-trimethylpiperazine for improving cognition. Moreover the invention relates to an improved pharmaceutical composition comprising 4-((1R,3S)-6-chloro-3-phenylindan-1-yl)-1,2,2-trimethylpiperazine.

### BACKGROUND OF THE INVENTION

The compound which is the subject of the present invention (Compound I, trans-4-((1R,3S)-6-chloro-3-phenylindan-1-yl)-1,2,2-trimethylpiperazine) has the formula (I):

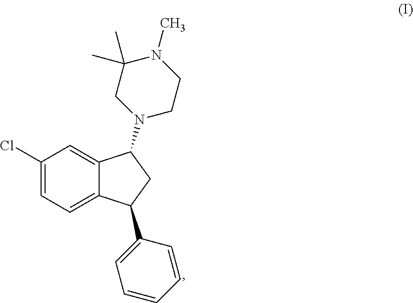

International patent publication No. WO 2005/016900 discloses the compound (i.e., Compound I) as a free base and its corresponding succinate and malonate salts. The compound is reported to have high affinity for dopamine D₁ (antagonist) and D₂ receptors (antagonist), the 5-HT₂ receptor (antagonist) and for α₁ adrenoceptors. In WO 2005/016900, the compound is disclosed to be useful for treatment of several diseases in the central nervous system, including psychosis, in particular schizophrenia (positive, negative, and/or depressive symptoms) or other diseases involving psychotic symptoms, such as, e.g., Schizophrenia, Schizophreniform Disorder, Schizoaffective Disorder, Delusional Disorder, Brief Psychotic Disorder, Shared Psychotic Disorder, as well as other psychotic disorders or diseases with psychotic symptoms, e.g., mania in bipolar disorder. Also disclosed in WO 2005/016900 is the use of Compound I for the treatment of anxiety disorders, affective disorders including depression, sleep disturbances, migraine, neuroleptic-induced parkinsonism, or cocaine abuse, nicotine abuse, alcohol abuse and other abuse disorders, and for the maintenance of bipolar disorders.

Other publications disclosing Compound I and related compounds, including the above pharmacological profile, are EP 638 073; Bøgesø K. P. et al. J. Med. Chem., 1995, 38, page 4380-4392; and Bøgesø K. P. “Drug Hunting, the Medicinal Chemistry of 1-Piperazino-3-phenylindans and Related Compounds”, 1998, ISBN 87-88085-10-4 (cf. e.g., compound 69 in table 3, p 47 and in table 9A, p 101).

The present inventors have now surprisingly found that Compound I, i.e., trans-4-((1R,3S)-6-chloro-3-phenylindan-1-yl)-1,2,2-trimethylpiperazine, has cognitive enhancing properties, and thus, the present invention is direct to this and other important ends.

### SUMMARY OF THE INVENTION

The invention relates to methods of treating cognitive dysfunction, such as, e.g., cognitive dysfunction associated with a certain disease, comprising the administration of Compound I or a pharmaceutically acceptable salt thereof to a patient in need thereof. The pharmaceutically acceptable salt of Compound I may be in the form of a pharmaceutical composition.

The invention also relates to the use of Compound I or a pharmaceutically acceptable salt thereof in the manufacture of a medicament for the treatment of cognitive dysfunction, such as, cognitive dysfunction associated with a certain disease.

In a further aspect the invention relates to an improved pharmaceutical composition comprising Compound I particularly useful for the treatment of cognitive dysfunction associated with a certain disease, such as schizophrenia.

### FIGURES

FIG. 1: Effects of Compound I in a rat disease model for schizophrenia with cognitive deficits: executive functioning in accordance with an embodiment of the present invention.

FIG. 2: Effects of Compound I in a rat disease model for schizophrenia with cognitive deficits: visual learning and memory (acquisition) in accordance with an embodiment of the present invention.

FIG. 3: Effects of Compound I in a rat disease model for schizophrenia with cognitive deficits: visual learning and memory (retention) in accordance with an embodiment of the present invention.

FIG. 4: Effects of Compound I in a rat disease model for schizophrenia with cognitive deficits: visual learning and memory (discrimination index) in accordance with an embodiment of the present invention.

FIG. 5: Effects of Compound I in a rat disease model for schizophrenia with cognitive deficits: visual learning and memory (locomotor activity) in accordance with an embodiment of the present invention.

FIG. 6: Flow diagram of the manufacturing process of film coated tablets and process controls.

### DETAILED DESCRIPTION OF THE INVENTION

Diminished cognitive processes (i.e., cognitive impairment, cognitive deficit, cognitive dysfunction and the like) can be experienced in several patient groups, e.g., in schizophrenic, depressive or psychotic patients and in patients with Parkinson's disease.

Cognitive impairment includes a decline in cognitive functions or cognitive domains, such as, e.g., difficulties with attention, learning, memory and executive function (relevant reactions to external stimuli). Cognitive impairment also may include: deficits in attention, disorganized thinking, slow thinking, difficulty in understanding, poor concentration, impairment of problem solving, poor memory, difficulty in expressing thoughts and/or integrating thoughts, feelings and behaviour, and/or extinction of irrelevant thoughts, and difficulty in attention and vigilance, verbal learning and memory, visual learning and memory, speed of processing, social cognition, reasoning and problem solving, e.g., executive functioning. There are presently no effective drugs for the treatment of cognitive disorders on the market and there is a great need and demand for drugs effective in the treatment of such disorders.

Cognitive deficits, including impairments in areas such as memory, attention, and executive function, are a major determinant and predictor of long-term disability in schizophrenia. Unfortunately, presently available antipsychotic medications are relatively ineffective in improving cognition.

Schizophrenia is characterized by three broad types of symptom groups, namely, positive symptoms (e.g., hallucinations), negative symptoms (e.g., affective blunting and social withdrawal), and impairments in information processing and cognitive functions (such as, e.g., executive functioning, attention and memory). Executive functioning incorporates processes such as planning, organization, mental flexibility and task coordination and is considered to be the domain in which schizophrenia patients have the most difficulties. Cognitive deficits in schizophrenia are also termed “cognitive impairment associated with schizophrenia” (CIAS). Yet cognitive impairment is observed in many patients prior to onset of psychotic symptoms and/or other clinical features. Furthermore, there is a close link between cognitive impairment and community functioning and unfavorable outcome in patients, and no efficacious treatment of these symptoms has been found yet.

The MATRICS (Measurement and Treatment Research to Improve Cognition in Schizophrenia) initiative in the USA between the National Institute of Mental Health, the University of California, Los Angeles, and the United States Food and Drug Administration, aiming at creating a consensus regarding the nature of cognitive impairments in schizophrenia and how they might be best assessed and treated, has identified seven critical domains of cognition including working memory, attention and vigilance, executive functioning (i.e., reasoning and problem solving), verbal learning, visual learning, speed of processing and social cognition. The current antipsychotics largely treat the positive symptoms of schizophrenia and have limited impact on the negative or cognitive symptoms. Furthermore, many antipsychotics currently on the market even provoke drug induced cognitive impairments. Therefore, there is a real need to develop better therapies to improve the cognitive dysfunction associated with schizophrenia.

The present inventors have now found that trans-4-((1R,3S)-6-chloro-3-phenylindan-1-yl)-1,2,2-trimethylpiperazine attenuates the attentional performance impairment induced in an animal model, indicating cognition enhancing properties of this compound (see e.g., Example 1 herein). The attentional set-shifting paradigm is an animal model that allows assessment of executive function via intra-(ID) versus extra-dimensional (ED) shift discrimination learning, and is functionally analogous to a sensitive test of frontal function in humans, viz. the Wisconsin Card Sorting Test (WCST) or the computerized intra-dimensional—extra-dimensional test. Specifically, this task requires rats to solve a series of discrimination problems by distinguishing which of two pots presented contains food rewards based on two or three non-spatial cue dimensions (odour, digging medium, and/or texture). A schizophrenia-disease-like animal model with subchronic phencyclidine (PCP) administration plus washout period is applied. The subchronic PCP with washout treatment regime appears to induce the most selective impairment, with a performance deficit confined to ED shift performance only; thus, indicating that this specific pharmacological manipulation may model more effectively the executive function deficits observed in first-episode schizophrenia patients.

Further, the present inventors have surprisingly found that trans-4-((1R,3S)-6-chloro-3-phenylindan-1-yl)-1,2,2-trimethylpiperazine attenuates the visual learning and memory impairment induced in an animal model, also indicating cognition enhancing properties of this compound (see e.g., Example 3 herein).

Thus, the overall findings of the above rat attentional set shifting test and the novel object recognition tests are indicative of trans-4-((1R,3S)-6-chloro-3-phenylindan-1-yl)-1,2,2-trimethylpiperazine having cognition enhancing properties.

Additionally trans-4-((1R,3S)-6-chloro-3-phenylindan-1-yl)-1,2,2-trimethylpiperazine is expected to be useful in the treatment of deficiencies relating to sensory gating, which is well known to be disturbed in Schizophrenia (see e.g. Adler, L. E. et al Schizophrenia Bulletin, Vol. 24, No. 2, 1998, page 189-202). Sensory gating is a process by which the brain adjusts its response to stimuli. It is a largely automatic process. When one stimulus is presented, there is a response. But when it is followed by a second stimulus soon after, the response to the second stimulus is blunted. This is an adaptive mechanism to prevent over stimulation. It helps the brain focus on a stimulus among a host of other distractors. The mechanism of sensory gating involves feed-forward and feed-back inhibition of the stimulus perceived. It involves GABA-ergic and a7 nicotinergic receptor-mediated inhibition of the pyramidal neurons in the cornu ammonis (CA3) region of the hippocampus.

Moreover, the present inventors have surprisingly found that in addition to its already known pharmacological profile, trans-4-((1R,3S)-6-chloro-3-phenylindan-1-yl)-1,2,2-trimethylpiperazine shows a potent in vitro antagonistic effect at 5-HT₆ receptors, which is a receptor target that has been associated with cognitive enhancing effects in both normal and disease states. (see e.g., Example 2 herein). This is based on non-clinical studies showing that treatment with 5-HT₆ antisense oligonucleotides, as well as 5-HT₆ antagonists, have cognition enhancing potential (Mitchell E S, Neumaier J F. “5-HT6 receptors: a novel target for cognitive enhancement.” Pharmacol Ther. 2005; 108:320-33). Like 5-HT₆ antagonists, Compound I reverses deficits in ED shift performance induced by PCP in rats, which indicates the pro-cognitive potential of the compound.

Further, the present inventors have found that Compound I is therapeutically effective in low doses, such as in an amount of 4 to 14 mg calculated as the free base.

The compound of formula I is a putative antipsychotic compound with affinity for both dopamine D1 and D2 receptors. Preclinical experiments in rats using the condition avoidance response (CAR) model (Experimental procedure previously described in: Hertel P, Olsen C K, Arnt J. Repeated administration of the neurotensin analogue NT69L induces tolerance to its suppressant effect on conditioned avoidance behaviour. Eur J Pharmacol. 2002; 439(1-3):107-11) have indicated that the compound of formula I possesses antipsychotic activity at very low levels of D2 receptor occupancy.

In a positron emission tomography (PET) study in healthy subjects using 11C-SCH23390 and ¹¹C-raclopride as D1 and D2 receptor tracers, it was found that the compound of formula I induces a D2 receptor occupancy of from 11 to 43% in the putamen when increasing the dose from 2 to 10 mg/day given daily for 18 days. Such level of D2 receptor occupancy is low in comparison with that of currently used antipsychotic drugs, which in general requires a D2 receptor occupancy around or exceeding 50% to be therapeutically effective (Stone J M, Davis J M, Leucht S, Pilowsky L S. Cortical Dopamine D2/D3 Receptors Are a Common Site of Action for Antipsychotic Drugs; An Original Patient Data Meta-analysis of the SPECT and PET In Vivo, Schizophr Bull. 2008 Feb. 26. [Epub in advance of print]). In the same PET study, it was found that the compound of formula I induces a D1 receptor occupancy increase from 32 to 69% in putamen when increasing the dose from 2 to 10 mg/day given daily for 18 days. Such high level of D1 occupancy is not generally seen with current used antipsychotic drugs (Farde L, Nordström A L, Wiesel F A, Pauli S, Halldin C, Sedvall G. Positron emission tomographic analysis of central D1 and D2 dopamine receptor occupancy in patients treated with classical neuroleptics and clozapine. Relation to extrapyramidal side effects. Arch Gen Psychiatry. 1992; 49(7):538-44.). Thus, the compound of formula I exhibits a unique ratio of D1 to D2 receptor occupancy at low daily doses.

Based on the above, it is expected that the compound of formula I have clinically significant therapeutic effects in patients with cognitive impairment and/or sensory gating and/or schizophrenia, in particular cognitive impairment and/or sensory gating in association with schizophrenia at doses (from 2mg/day to 14 mg/day, in particular 4 mg/day to 14 mg/day) that induce only a low level of D2 receptor occupancy. This might well be a consequence of the high D1 receptor occupancy and the unique ratio of D1 versus D2 receptor occupancy displayed by the compound of formula I. A low D2 receptor occupancy at therapeutically effective doses will be beneficial in terms of reduced tendency to induce troublesome side effects mediated by D2 receptor blockade, including extrapyramidal side effects and hyperprolactinemia.

The compound of formula I in a therapeutically effective amount of from 2-14 mg, in particular 4-14 mg calculated as the free base is administered orally, and may be presented in any form suitable for such administration, e.g. in the form of tablets, capsules, powders, syrups or solutions. In one embodiment, a salt of the compound of formula I is administered in the form of a solid pharmaceutical entity, suitably as a tablet or a capsule.

Methods for the preparation of solid pharmaceutical compositions or preparations are well known in the art. Thus, tablets may be prepared by mixing the active ingredient with conventional adjuvants, fillers and diluents and subsequently compressing the mixture in a suitable tabletting machine. Examples of adjuvants, fillers and diluents comprise cornstarch, lactose, talcum, magnesium stearate, gelatine, gums, and the like. Typical fillers are selected from lactose, mannitol, sorbitol, cellulose and microcrystalline cellulose. Any other adjuvant or additive such as colourings, aroma, preservatives, etc, may also be used provided that they are compatible with the active ingredient.

Accordingly, the present invention relates to certain pharmaceutical uses of trans-4-((1R,3S)-6-chloro-3-phenylindan-1-yl)-1,2,2-trimethylpiperazine (Compound I) or a pharmaceutically acceptable salt thereof or a pharmaceutical composition comprising said salt.

The compound of formula I (or Compound I) as used throughout the present description is intended to designate any form of the compound, such as the free base, pharmaceutically acceptable salts thereof, e.g. pharmaceutically acceptable acid addition salts, such as succinate and malonate salts, hydrates or solvates of the free base or salts thereof, as well as anhydrous forms, amorphous forms, or crystalline forms.

The compound of formula I to be comprised in the composition of the present invention also comprises salts thereof, typically, pharmaceutically acceptable salts. Such salts include pharmaceutical acceptable acid addition salts. Acid addition salts include salts of inorganic acids as well as organic acids. Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like.

Further, the compound of formula I may exist in unsolvated form, as well as in solvated forms with pharmaceutically acceptable solvents such as water, ethanol and the like. In general, solvated forms are considered to be equivalent to unsolvated forms for the purposes of this invention.

In a further embodiment of the composition, the use, or the method of treatment, the composition further comprises povidone or copovidone, such as Kollidone VA64, as a binder. The binder is typically present in a concentration range of from 2-10% (w/w), such as 2-4%, 4-6%, 6-8%, 8-10%, 2-8%, 4-8%, 4-10%, or 6-10% (w/w).

In a further aspect the present invention also relates to a pharmaceutical composition comprising the compound of formula (I) and povidone or copovidone as binder. Typically the binder is Kollidone VA64. In a particular embodiment the said pharmaceutical composition is for the treatment of cognitive impairment or schizophrenia, particularly for the treatment of cognitive impairment in association with schizophrenia.

In an embodiment the binder is present in a concentration range of from 2-10% (w/w), typically in a concentration range of from 2-4%, 4-6%, 6-8%, or 8-10% (w/w). When the binder is povidone or copovidone typical fillers are selected from calcium hydrogen phosphate lactose, mannitol, sorbitol, cellulose and microcrystalline cellulose, and preferably lactose, mannitol, sorbitol, cellulose and microcrystalline cellulose, such as lactose. In an embodiment the filler, such as anyone of the above, is in a concentration range of from 15-50% (w/w). Typically, the filler, such as anyone of lactose, mannitol, sorbitol, cellulose and microcrystalline cellulose, is in a concentration range of from 15-25%, 20-50%, 30-45% (w/w).

As used herein, the phrases “cognitive deficit(s)”, “cognitive impairment(s)”, and “cognitive dysfunction(s)” are intended to indicate the same and are used interchangeably. As such these phrases refer to the interference or disruption of one or more cognitive processes, cognitive functions and/or cognitive domains. In some instances, “cognitive deficit(s)”, “cognitive impairment(s)”, and “cognitive dysfunction(s)” are related to and/or are associated with one or more functional impairments that often result in poor social/community adaptation and work disability.

In another aspect, the invention relates to a method of improving cognitive functioning, comprising administering an effective amount of Compound I or a pharmaceutically acceptable salt thereof to a patient in need thereof.

In the present context, the terms “improves”, “improving”, and the like, mean to make better; to enhance. In some instances, the term refers to an enhancement of cognitive performance as based on a consensus battery as an endpoint (e.g., MATRICS Consensus Cognitive Battery's overall composite score, which is composed of equal weighting of the seven domain scores, as a primary endpoint in measuring improved cognitive functioning).

The invention also relates to a method of treating cognitive dysfunction, comprising administering an effective amount of Compound I or a pharmaceutically acceptable salt thereof to a patient in need thereof.

In the present context, the terms “treatment”, “treating”, and the like, mean the management and care of a patient for the purpose of combating a disease, disorder or condition (herein, and without limitation, a cognitive dysfunction). The term is intended to include the full spectrum of treatments for a given disease, disorder or condition as described herein from which the patient is suffering, such as administration of the active compound to alleviate or relieve a symptom(s) or complication(s) of the disease, disorder or condition, to delay the progression of the disease, disorder or condition, as well as to prevent the disease, disorder or condition, wherein prevention is to be understood as the management and care of the patient for the purpose of combating the disease, condition, or disorder and includes the administration of the active compound to prevent the onset of the symptom(s) or complication(s). The terms “treatment”, “treating”, and the like, also mean to cure or eliminate the disease, disorder or condition. Nonetheless, prophylactic (preventive) and therapeutic (curative) treatments are two separate aspects of the invention. The patient to be treated is, e.g., a mammal, such as a human being.

As used herein, the phrase “effective amount” when applied to a compound of the invention, is intended to denote an amount sufficient to cause an intended biological effect. The phrase “therapeutically effective amount” when applied to a compound of the invention is intended to denote an amount of the compound that is sufficient to ameliorate, palliate, stabilize, reverse, slow or delay the progression of a disease, disorder or condition state, or of a symptom of the disease, disorder or condition.

In another aspect, the invention relates to Compound I or a pharmaceutically acceptable salt thereof for use in a method of the present invention, wherein the method is for improving cognitive functioning, such as and without limitation, in a patient suffering from a cognitive dysfunction.

In another aspect, the invention relates to the use of Compound I or a pharmaceutically acceptable salt thereof for the preparation of a medicament for improving cognitive functioning, such as and without limitation, in a patient that has (i.e., suffers from) a cognitive dysfunction. The invention also relates to the use of Compound I or a pharmaceutically acceptable salt thereof for the preparation of a medicament for treating cognitive dysfunction.

The invention further provides Compound I or a pharmaceutically acceptable salt thereof for the treatment of a cognitive dysfunction in a disease selected from the group consisting of schizophrenia, a disease involving psychotic symptoms, schizophreniform disorder, schizoaffective disorder, delusional disorder, brief psychotic disorder, shared psychotic disorder and substance-induced psychotic disorder, an affective disorder (such as, e.g., depression, bipolar disorder and mania), Parkinson's disease, a disease involving a sleep disturbance, neuroleptic-induced parkinsonism, and an abuse disorder (such as, e.g., cocaine abuse, nicotine abuse, and alcohol abuse).

The invention further provides a method of treating cognitive impairment associated with schizophrenia (CIAS), comprising administering a therapeutically effective amount of trans-4-((1R,3S)-6-chloro-3-phenylindan-1-yl)-1,2,2-trimethylpiperazine or a pharmaceutically acceptable salt thereof to a patient in need thereof.

The invention further provides a pharmaceutical composition comprising a therapeutically effective amount of Compound I or a pharmaceutically acceptable salt thereof and a pharmaceutically acceptable adjuvant, filler, diluent, additive, or combination thereof, for a use as described herein.

In one embodiment of the invention, the pharmaceutically acceptable salt is a succinate salt or a malonate salt. In one embodiment, the pharmaceutically acceptable salt is in the form of a crystalline hydrogen succinate salt of Compound I or a crystalline hydrogen malonate salt of Compound I, e.g., crystal form alpha of the hydrogen succinate salt of Compound I or crystal form alpha of the hydrogen malonate salt of Compound I. The succinate salt and malonate salt of Compound I and their preparations are described in WO 2005/016900.

In one embodiment of the present invention, Compound I or pharmaceutically acceptable salt thereof is in a purified form. The term “purified form” is intended to indicate that Compound I or salt thereof is essentially free of other compounds or other forms of the compound (such as polymorphic forms), as the case may be.

In one embodiment, the patient of the invention is suffering from a cognitive dysfunction. In one embodiment of the invention, the patient is not suffering from a cognitive dysfunction. In one embodiment, the patient of the invention is a first-episode schizophrenia patient. In one embodiment, the patient of the invention has been diagnosed with a cognitive impairment for which the patient is being treated.

In one embodiment, the cognitive dysfunction of the invention is in connection with a disease. In one such embodiment, the disease is selected from the group consisting of a disease involving a psychotic symptom (such as, e.g., schizophrenia), schizophreniform disorder, schizoaffective disorder, delusional disorder, brief psychotic disorder, shared psychotic disorder, substance-induced psychotic disorder, an affective disorder (such as, e.g., depression, bipolar disorder and mania), Parkinson's disease, a disease involving a sleep disturbance, neuroleptic-induced parkinsonism, and an abuse disorder (such as, e.g., cocaine abuse, nicotine abuse, and alcohol abuse).

In one embodiment, the method of the invention comprises administering an effective amount of Compound I or a pharmaceutically acceptable salt thereof to a patient in need thereof.

In one embodiment of the invention, Compound I or a pharmaceutically acceptable salt thereof is used for treating cognitive dysfunction in connection with schizophrenia. In one embodiment, the cognitive dysfunction is CIAS. In one embodiment, the use reduces a cognitive symptom in a schizophrenic patient. In one embodiment, the patient has at least one cognitive symptom of schizophrenia. In one embodiment, the patient has two or more cognitive symptoms of schizophrenia. As used herein, the phrase “cognitive symptom(s)” refers to the cognitive deficit(s), cognitive dysfunction(s) and cognitive impairment(s) often associated with schizophrenia. As used herein, the terms “reduces, “reducing” and the like, refers to a lessening or diminishing, such as, e.g., in severity, effect, and presence.

In a further embodiment of the invention, the method of treating a cognitive impairment associated with a disease as described herein, e.g., schizophrenia, further comprises wherein the cognitive impairment is manifested as a decline in at least one function or domain selected from the group consisting of working memory, attention, verbal learning and memory, problem solving (e.g., executive function), speed of processing and social cognition.

In a further embodiment of the invention, the cognitive dysfunction(s) (i.e., cognitive impairment(s), cognitive dysfunction(s)) to be treated include a decline in a cognitive function or cognitive domain, e.g., one selected from the group consisting of working memory, attention and vigilance, verbal learning and memory, visual learning and memory, reasoning and problem solving (e.g., executive function), speed of processing, social cognition, and a combination thereof, such as attentional performance in combination with visual learning and memory. Also, cognitive deficits, cognitive impairment and the like, may indicate deficits in attention, disorganized thinking, slow thinking, difficulty in understanding, poor concentration, impairment of problem solving, poor memory, deficits in planning, organization, deficits in mental flexibility, deficits in task coordination, difficulties in expressing thoughts, difficulties in integrating thoughts, feelings and behaviour, difficulties in extinction of irrelevant thoughts, or a combination thereof.

Synthesis of Compound I, including Definitions

Compound I, including the succinate and malonate salts thereof, may be prepared as outlined in WO 2005/016900.

It is understood that when specifying the stereoisomeric form, the stereoisomer is the main constituent of the compound. For example, when specifying an enantiomeric form of the compound, the compound has an enantiomeric excess of the enantiomeric form specified.

In the present invention, for the pharmaceutical uses it is understood that when specifying the enantiomeric form of the compound trans-4-(6-chloro-3-phenylindan-1-yl)-1,2,2-trimethylpiperazine, as done in formula (I), the compound is relatively stereochemically pure, e.g., the enantiomeric excess is of at least about 70%, at least about 80%, at least about 90%, at least about 96%, or at least about 98%, where, for example, an “enantiomeric excess if at least about 80%” means that the ratio of Compound I to its enantiomer is 90:10 in the compound mixture in question. In one embodiment, the diastereomeric excess of Compound I (i.e., the cis/trans ratio) is at least about 90%, at least about 95%, at least about 97%, or at least about 98%, where, for example, 90% diastereomeric excess means that the ratio of Compound I to cis-4-((1S,3S))-6-chloro-3-phenylindan-1-yl)-1,2,2-trimethylpiperazine is 95:5.

The enantiomeric excess of Compound I may, e.g., be determined as described in WO 2005/016900, which briefly is by fused silica capillary electrophoresis (CE) using the following conditions: Capillar: 50 μm ID×64.5 cm L, run buffer: 1.25 mM β cyclo dextrin in 25 mM sodium dihydrogen phosphate, pH 1.5, voltage: 16 kV, temperature: 22° C., injection: 50 mbar for 5 seconds, detection: column diode array detection 192nm, sample concentration: 500 μg/ml. In this system, Compound I has a retention time of approximately 33 min, and the other enantiomer has a retention time of approximately 35 min. The diastereomeric excess of Compound I may, e.g., be determined as described in Bøgesø et al., J. Med. Chem. 1995, 38, 4380-4392 (page 4388, right column)

In the present invention, pharmaceutically acceptable salts include any pharmaceutically acceptable salt of Compound I. Non-limiting examples of such salts are crystalline hydrogen succinate salt and crystalline malonate salt of Compound I.

### Administration and Dose of Compound I:

Compound I or a salt thereof may be administered in any suitable way, e.g., orally, buccal, sublingual or parenterally, and the salt may be presented in any suitable form for such administration, e.g., in the form of tablets, capsules, powders, syrups or solutions or dispersions for injection. In one embodiment, a salt of the invention is administered in the form of a solid pharmaceutical entity, suitably as a tablet or a capsule.

Methods for the preparation of solid pharmaceutical preparations are well known in the art. Tablets may thus be prepared by mixing the active ingredient with ordinary adjuvants, fillers and diluents and subsequently compressing the mixture in a convenient tabletting machine. Non-limiting examples of adjuvants, fillers and diluents comprise cornstarch, lactose, talcum, magnesium stearate, gelatine, lactose, gums, and the like. Any other adjuvant or additive, such as colourings, aroma, preservatives, etc., may also be used provided that they are compatible with the active ingredients.

Solutions for injections may be prepared by dissolving a salt of the invention and possible additives in a part of the solvent for injection, such as sterile water, adjusting the solution to desired volume, sterilisation of the solution and filling in suitable ampules or vials. Any suitable additive conventionally used in the art may be added, such as tonicity agents, preservatives, antioxidants, solubilising agents, etc.

The daily dose of Compound I calculated as the free base, is suitably between about 2 and about 55 mg, or between about 3 and about 55 mg. Accordingly, within the invention is a method of treating a cognitive impairment as described herein comprising administering Compound I or a pharmaceutically acceptable salt thereof to a patient in need thereof, wherein the daily dose of Compound I calculated as the free base, is between about 2 and about 55 mg, or between about 3 and about 55 mg.

In some embodiments of the composition, use, or method of treatment the amount of Compound I, calculated as the free base, is between about 4 mg and about 14 mg.

In further embodiments of the composition, use, or method of treatment, the amount of the compound of formula (I) is from about 4 mg to about 12 mg.

In further embodiments of the composition, use, or method of treatment, the amount of the compound of formula (I) is from about 5mg to about 14 mg.

In further embodiments of the composition, use, or method of treatment, the amount of the compound of formula (I) is from about 4 to about 6 mg, such as about 5 mg.

In further embodiments of the composition, use, or method of treatment, the amount of the compound of formula (I) is from about 6 to about 8 mg, such as about 7 mg.

In further embodiments of the composition, use, or method of treatment, the amount of the compound of formula (I) is from about 8 to about 10 mg.

In further embodiments of the composition, use, or method of treatment, the amount of the compound of formula (I) is from about 10 to about 12 mg.

In further embodiments of the composition, use, or method of treatment, the amount of the compound of formula (I) is from about 12 to about 14 mg, such as about 14 mg.

In further embodiments of the composition, use, or method of treatment, the amount of the compound of formula (I) is from about 5 to about 7 mg.

In further embodiments of the composition, use, or method of treatment, the amount of the compound of formula (I) is from about 7 to about 9 mg.

In further embodiments of the composition, use, or method of treatment, the amount of the compound of formula (I) is from about 9 to about 11 mg, such as about 10 mg.

In further embodiments of the composition, use, or method of treatment, the amount of the compound of formula (I) is from about 11 to about 13 mg.

When the invention relates to the use or the method of treatment then the dose indicated above of from about 4-14 mg, such as about 4 mg, about 5 mg, about 6 mg, about 7 mg, about 8 mg, about 9 mg, about 10 mg, about 11 mg, about 12 mg, about 13 mg or about 14 mg, is on a daily basis. In a preferred embodiment the dose is about 5 mg, about 7 mg or about 10 mg.

In a particular embodiment the dose is as low as about 2 or about 3 mg of Compound I on a daily basis.

The invention will be illustrated in the following non-limiting examples.

### EXAMPLES

### Example 1

### Rat Disease Model for Schizophrenia with Cognitive Deficits, Executive Functioning

Executive functioning includes processes such as planning, organization, mental flexibility and task coordination and is considered to be the domain in which schizophrenia patients have the most difficulties. The attentional set-shifting paradigm is an animal model which allows assessment of executive functioning via intra-dimensional (ID) versus extra-dimensional (ED) shift discrimination learning. A schizophrenia-disease-like animal model with subchronic phencyclidine (PCP) administration plus washout period is applied. The subchronic PCP with washout treatment regime appears to induce the most selective impairment, with a performance deficit confined to ED shift performance only, indicating that this specific pharmacological manipulation may model more effectively the executive functioning deficits observed in first-episode schizophrenia patients.

Accordingly, the cognitive enhancing properties of a compound can be investigated by testing whether the compound is attenuating the “attentional performance impairment” induced by subchronic PCP administration in rats.

Methodology. In vivo assessment of “attentional set-shifting performance” was conducted as described by Rodefer et al. (Eur. J. Neurosci. 21:1070-1076 (2005)), and was based on a modified version of the task designed by Birrell & Brown (J. Neurosci. 20:4320-4324 (2000)). Briefly, Male Long-Evans rats (Harlan, Indianapolis, Ind.) weighing approximately 250 g at the beginning of the study in four groups (n=12 each group) were used. After habituating to the colony room environment, rats received a series of subchronic injections of PCP (5 mg/kg, i.p.) or saline twice daily for 7 days, followed by a washout period of 10 days before beginning the set-shifting procedure in which animals were required to dig in a pot to retrieve a food reward (half of a Honey Nut Cheerio® (General Mills, Minneapolis, Minn., USA)) using either digging medium or scented odor as the dimensional cue. The test chamber was a Plexiglas® box (50×37.5×25 cm) with an opaque barrier separating one-third of the box from the rest (along the long axis of the box).

On each trial, the two digging pots were placed adjacent to each other in the large section of the box while the rat waited in the small section. The rat was given access to the pots by raising the divider, which was then put back down once the trial had begun.

Habituation was performed for several days before testing; a pot filled with wood chip bedding used in the rats' home cages and baited with several Cheerios® was placed in each rat's cage, to accustom them to retrieving food rewards from the pots. Next, rats were placed in the test box daily for several days and given access to two pots filled with wood chip bedding and baited with several Cheerios®. The cups were replenished with bait continuously until the rats were digging reliably to retrieve the food rewards. At the end of this period, rats were trained on two simple discrimination problems (SDs) to a criterion of six consecutive correct trials: a problem involving the dimension of odor and medium, respectively. In all discrimination problems, digging was defined as a vigorous displacement of the digging medium, because the reward was buried deeply (about 2.5 cm) within the pot. The rats therefore could investigate the digging medium with paws or snout before executing a “dig” response, and these choices were not scored. Because the rats were allowed to sample the digging media by touch before digging, they could have used tactile or visual characteristics (or both) of the media to make their choices based on this dimension. All media included a small amount of powdered Cheerios®, to discourage the rat from trying to detect the hidden reward by odour alone. The purpose of this preliminary phase was to acquaint the rats with the discrimination learning procedure and the two possible relevant dimensions of odor and medium. The total time spent in this training phase varied (approximately 5-7 training days, on average) depending on how quickly each animal learned to reliably dig vigorously in order to find the food rewards. This procedure was developed in order to ensure that all animals would reliably dig in a robust and consistent manner that would enable each rat to complete the entire task in one test session. Thus, the test session took place approximately at least 2-3 weeks following completion of subchronic dosing with PCP.

On the day of testing, a dose of trans-4-((1R,3S)-6-chloro-3-phenylindan-1-yl)-1,2,2-trimethylpiperazine (1.25 or 2.5 mg/kg, s.c.) or vehicle (5% a.q. solution of hydroxyl-propyl-beta-cyclodextrin) was administered to the rats 30 min before the test session. The first four trials of each discrimination constituted a discovery period in which the rat was allowed to dig in both pots regardless of where the rat first began to dig. An error was recorded if the rat first dug in the unbaited pot. After the first four trials were completed and the rat dug first in the unbaited pot, the rat was returned to the small section of the box and was not permitted to find the food reward in the other pot. Testing continued until the rat reached a criterion of six consecutive correct trials.

In a single test session rats performed a series of discriminations paralleling the procedure used previously (Birrell & Brown J. Neurosci. 20:4320-4324 (2000); Rodefer et al. Neuropsychopharmacol. 33:2657-2666 (2008)). Initially, a simple discrimination (SD) between either two odors or two digging media was presented, followed by a compound discrimination (CD) with the same positive stimulus as the initial SD. In the CD a new dimension was introduced, but it was not a reliable predictor of the location of the food reward. The CD problem was then reversed (Rev1), so that what was formerly the unreinforced stimulus was changed to be the reinforced stimulus, with the irrelevant dimension not predictive of the reward location. An intra-dimensional shift (IDS) discrimination problem was then presented; the IDS problem was a compound discrimination in which the specific stimuli within both relevant and irrelevant dimensions were changed, but the relevant dimension (either odor or medium) remained the same. The IDS problem was then reversed (Rev2), so that what was formerly the unreinforced stimulus was changed to be the reinforced stimulus and with the irrelevant dimension still not predictive of the location of the reward. Then the rats were presented with an extra-dimensional shift (EDS) problem in which the formerly irrelevant dimension became the relevant one and the originally relevant dimension no longer held predictive value. Finally, the EDS problem was reversed (Rev3) such that what was formerly the unreinforced stimulus was changed to be the reinforced stimulus.

Data Analysis. Based on previous data (Rodefer et al. Int J. Neuropsychopharmacol 9:S140-141 (2006); Rodefer et al. Eur. J. Neurosci. 21:1070-1076 (2005)), it was hypothesized that subchronic PCP administration would selectively impair EDS task performance. Thus, first examined was set-shifting performance (trials to criterion) of the rats treated with subchronic PCP or saline using an analysis of variance (ANOVA) with 2 between-subjects factors (discrimination problem, drug pre-treatment) and the problem×drug interaction. Bonferroni corrected post hoc analyses were subsequently used to test mean differences.

Description of Results.

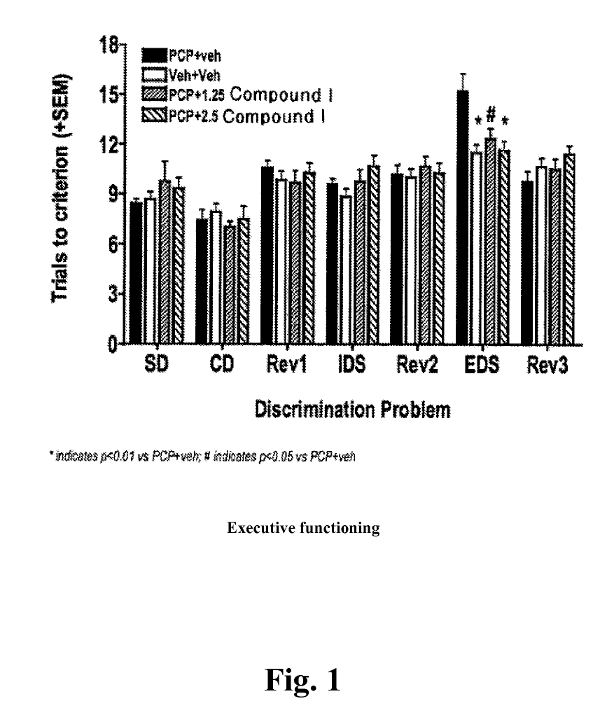

Effect of subchronic PCP treatment. Subchronic PCP-treatment produced a significant main effect for discrimination problem (F (6, 132)=22.06, p<0.01) but not a significant main effect for pre-treatment condition (F (1, 132)=2.92, p>0.05). The interaction between pre-treatment condition and discrimination problem was significant (F (6, 132)=4.04, p<0.01). Bonferroni post hoc analyses revealed a significant difference between PCP and saline treated animals only in the trials required to reach criterion in the EDS discrimination problem (t=4.51, p<0.01) (see FIG. 1). There were no suggestions of PCP-induced impairment on trials to criterion on any of the other discrimination problem (all ps>0.05). Thus, these data supported our hypothesis that subchronic PCP-administration would selectively impair EDS task performance

Effects of Compound I. Next tested were the effects of Compound I dose on reversing the PCP-induced deficit in EDS function (see immediately above). These analyses revealed a significant main effect of discrimination problem (F (6, 198)=20.54, p<0.01) but not of Compound I dose (F (2, 198)=0.23, p>0.05). However, there was a significant interaction between Compound I dose and discrimination problem (F(12,198) =2.07, p<0.05). Bonferroni post hoc comparisons revealed significant differences (see FIG. 1) on EDS discrimination performance between the PCP+vehicle group and both the group treated with 1.25 mg/kg Compound I (t=3.09, p<0.05) and the group treated with 2.5 mg/kg Compound I (t=3.80, p<0.01). No evidence was found that either dose of Compound I impacted behavior on all other discrimination problems (all ps>0.05). Thus, Compound I significantly attenuated the PCP-induced deficit in EDS learning across both doses examined

Effects of drug on time to complete task. Apparently Compound I produced a delay in task completion. A one-way ANOVA (F (2, 33)=11.95, p<0.01) revealed that these time differences were significant. Rats that received 2.5 mg/kg of Compound I (M=3.33 hrs, SD=1.5) took on average significantly longer to complete the set-shifting task than did the rats that received 1.25 mg/kg of Compound I (M=2.05 hrs, SD 1.1; t=2.89, p<0.05) or vehicle treated rats (M=1.18 hrs, SD=0.25; t=4.86, p<0.01). Rats that received 1.25 mg/kg Compound I did not differ significantly from vehicle treated rats (t=1.97, p>0.05). Observations suggested that Compound I treated rats performed in bursts of activity with pauses during the testing period. Thus, despite significantly attenuating the PCP-induced deficit, selected doses of drug also produced increased session duration. Yet, these increased times did not impact overall accuracy in EDS or other task phases. Moreover, animals with longer test session periods have been observed previously with aged animals, with animals that had received lesions that impacted motor coordination, and following administration of pharmacological compounds.

In contrast to clozapine, risperidone and haloperidol, which have been shown to be ineffective in reversing PCP-disrupted attentional performance when given acutely (see e.g., Rodefer et al. (2008)), trans-4-((1R,3S)-6-chloro-3-phenylindan-1-yl)-1,2,2-trimethylpiperazine was found to significantly attenuate the PCP-induced deficit in EDS learning across both doses examined Although select doses of trans-4-((1R,3S)-6-chloro-3-phenylindan-1-yl)-1,2,2-trimethylpiperazine appear to increase the amount of time for completing a task, the increased times did not affect the accuracy in EDS or other task phases of test and such increased times may be due to other unknown variables. Thus, the overall findings of the above rat attentional set shifting test are indicative of trans-4-((1R,3S)-6-chloro-3-phenylindan-1-yl)-1,2,2-trimethylpiperazine having cognition enhancing properties.

### Example 2

### In Vitro Antagonistic Effect of Compound I at 5-HT₆ Receptors

5-HT₆ receptors have been associated with cognitive enhancing effects in both normal and disease states. The in vitro antagonistic effect of trans-4-((1R,3S)-6-chloro-3-phenylindan-1-yl)-1,2,2-trimethylpiperazine (Compound I) at 5-HT₆ receptors was assessed by the following radioligand binding assay.

HeLa cells stably transfected with the human 5-hydroxytryptamine receptor 6 (NHI, November 1994) were cultured in screening plates. When reaching confluence (5-7 days), cells were harvested in ice cold D-PBS (Dulbecco's Phosphate-Buffered Saline) using a cell scraper, centrifuged at 1000 rpm for 10 minutes and re-suspended in 1 mL D-PBS per plate. Cell membranes were stored at −80° C.

Before the experiment, membranes were quickly thawed and homogenized in ice cold 50 mM TRIS buffer pH 7.7, using an Ultra-Turrax® homogenizer (IKA® Werke GmbH & Co. KG, Staufen, Germany). Also before the experiment, all test compounds were diluted in 50 mM TRIS buffer pH 7.7.

Aliquots consisting of 10 μL test compound/total/non-specific, 10 μL radio ligand, [³H]5-LSD ([N-methyl-³H]Lysergic acid, diethylamide) (#TRK1041, 1 nM final; GE Healthcare, Hillerød, Denmark, formerly Amersham Biosciences) and 180 μL membrane suspension (10 μg final) were incubated at RT for 30 minutes. Bound ligand was separated from free ligand by filtration on a Tomtec® Harvester 96 Mach 3u (Tomtec, Hamden, Conn.). Filters were washed 2 times with 0.5 mL ice-cold 50 mM TRIS buffer pH 7.7. The filters were dried 20 minutes (37° C.) before addition of OptiPhase SuperMix (Perkin Elmer Wallac, Gaithersburg, Md., USA) and counted in a MicroBeta® TriLux 1450 (Perkin Elmer Wallac, Gaithersburg, Md., USA) counter for 1 minute.

Total binding was determined using TRIS buffer and non-specific binding was determined using 10 μM 5-Fluoro-1-(4-fluorophenyl)-3-[1-[2-(2-imidazolidinon-1-yl)ethyl]-4-piperidyl]-1H-indole (H. Lundbeck A/S, Valby, Denmark).

IC₅₀ values were determined by non-linear curve fitting using XlFit (IDBS), and Kᵢ values were calculated from the Cheng-Prusoff equation:

Kᵢ=IC₅₀/(1−[L]/K_{D}),

where [L] is the concentration of radioligand and K_{D} is its dissociation constant at the receptor, derived from the saturation isotherm.

The Kᵢ value for three different batches of Compound I were 0.78 nM, 1.4 nM and 0.84 nM. Compound I therefore shows a potent in vitro antagonistic effect at 5-HT₆ receptors, indicating a cognitive enhancing effect.

### Example 3

### Rat Disease Model for Schizophrenia with Cognitive Deficits

It has been demonstrated (Grayson B. et al. Behavioural Brain Research 184 (2007) 31-38) that sub-chronic PCP (phencyclidine) treatment in combination with the novel object recognition (NOR) test is a useful model for detecting compounds with therapeutic potential in treating the symptomology of cognitive dysfunction associated with schizophrenia.

The NOR test was performed as described in the above cited reference (Grayson 2007).

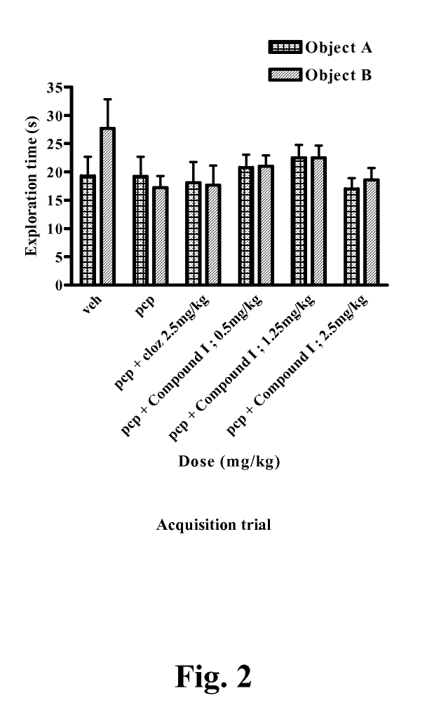

Acquisition trial. All groups of rats spent equivalent time exploring the identical objects (A and B) in the acquisition phase. FIG. 2 shows the mean exploration time of identical objects in the acquisition phase of the novel object recognition task (NOR) following acute trans-4-((1R,3S)-6-chloro-3-phenylindan-1-yl)-1,2,2-trimethylpiperazine (0.5-2.5 mg/kg, s.c.) or clozapine (2.5 mg/kg, i.p.) in sub-chronic PCP and vehicle treated rats. Data are expressed as mean±s.e.m. (n=9-10 per group) and were analysed by ANOVA and post-hoc student's t-test. Statistical analysis showed no significant difference in time spent exploring the identical objects in the acquisition phase in any group.

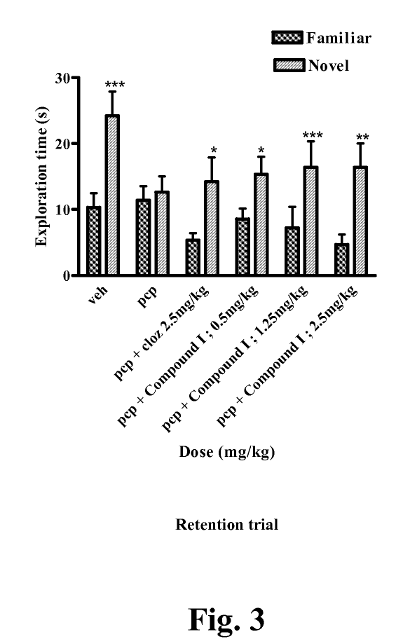

Retention trial. FIG. 3 shows mean exploration time of familiar and novel objects in the retention phase of the novel object recognition task (NOR) following acute trans-4-((1R,3S)-6-chloro-3-phenylindan-1-yl)-1,2,2-trimethylpiperazine (0.5-2.5 mg/kg, s.c.) or clozapine (2.5 mg/kg, i.p.) in sub-chronic PCP and vehicle treated rats. Data are expressed as mean±s.e.m. (n=9-10 per group) and were analysed by ANOVA and post-hoc student's t-test. Statistical analysis showed a significant difference between time spent exploring the familiar and the novel object *P<0.05-***P<0.001.

Rats treated sub-chronically with the vehicle spent significantly (p<0.001) longer time exploring the novel object compared with the familiar object (FIG. 3). The ability to discriminate familiar and novel objects was abolished following sub-chronic PCP treatment, whereby there was no significant difference in exploration of the novel and the familiar objects (FIG. 3).

Acute treatment with clozapine at a dose of 2.5 mg/kg significantly attenuated the sub-chronic PCP-induced impairment such that a significant increase in time spent exploring the novel compared with the familiar object was again observed (p<0.05).

Acute treatment with trans-4-((1R,3S)-6-chloro-3-phenylindan-1-yl)-1,2,2-trimethylpiperazine at all three doses (0.5 mg/kg-1.25 mg/kg-2.5 mg/kg) significantly attenuated the sub-chronic PCP-induced impairment such that a significant increase in time spent exploring the novel compared with the familiar object was again observed. The attenuation was even more pronounced with treatment with trans-4-((1R,3S)-6-chloro-3-phenylindan-1-yl)-1,2,2-trimethylpiperazine than it was with treatment with clozapine.

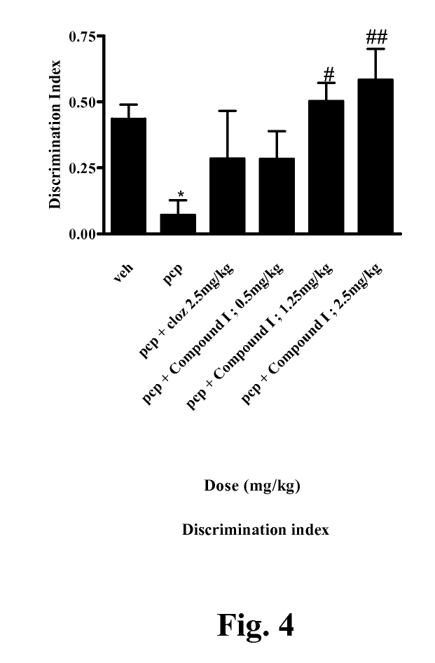

FIG. 4 shows the effect of administration with trans-4-((1R,3S)-6-chloro-3-phenylindan-1-yl)-1,2,2-trimethylpiperazine (0.5-2.5 mg/kg, s.c.) or clozapine (2.5 mg/kg, i.p.) on the discrimination index in sub-chronically PCP treated rats. Data are expressed as the mean±s.e.m. (n=9-10 per group) and were analysed using ANOVA followed by post-hoc Dunnett's t-test. The discrimination index was significantly (p<0.05, compared with vehicle) reduced following sub-chronic PCP treatment, whereas this effect was significantly (p<0.05-p<0.01, compared with PCP) attenuated upon treatment with trans-4-((1R,3S)-6-chloro-3-phenylindan-1-yl)-1,2,2-trimethylpiperazine at two of the doses (1.25 mg/kg-2.5 mg/kg). This is in contrast to treatment with clozapine where no significant effect of the treatment (2.5 mg/kg) is observed.

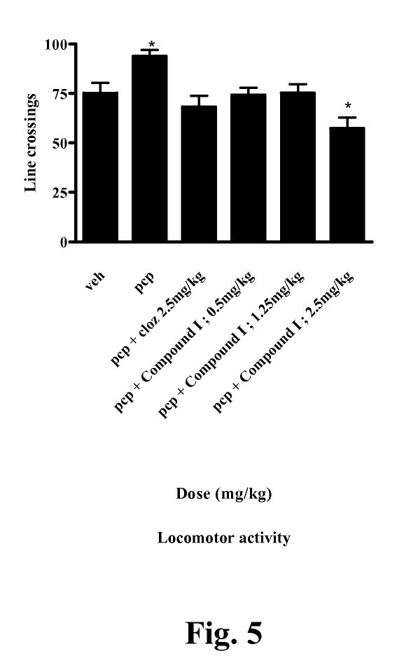

Locomotor activity. FIG. 5 shows the effect of administration with trans-4-((1R,3S)-6-chloro-3-phenylindan-1-yl)-1,2,2-trimethylpiperazine (0.5-2.5 mg/kg, s.c.) or clozapine (2.5 mg/kg, i.p.) on sub-chronically PCP treated rats on the total number of line crossings in the novel object recognition task. Data are expressed as mean±s.e.m (n=9-10 per group) and were analysed by ANOVA and post-hoc Dunnett's t-test.

Rats treated sub-chronically with PCP showed significantly (p<0.05, compared to vehicle) higher locomotor activity, whereas the locomotor activity was significantly reduced (p<0.05, compared to vehicle) upon treatment with trans-4-((1R,3S)-6-chloro-3-phenylindan-1-yl)-1,2,2-trimethylpiperazine at the highest of the doses (2.5 mg/kg). No significant effect of the other treatments on locomotor activity was observed.

Thus, the overall findings of the above NOR tests are indicative of trans-4-((1R,3S)-6-chloro-3-phenylindan-1-yl)-1,2,2-trimethylpiperazine having cognition enhancing properties.

### Example 4

### Preparation of Immediate Release Film-Coated Tablet Intended for Oral Administration I

### Pharmaceutical Development

A study of the compatibility of the excipients and compound of formula I demonstrated that the components used in the tablet formulation were compatible with the compound. Based on this, a traditional wet granulation, tabletting and film-coating process was developed using standard methods and excipients.

### Description of Drug Product

The compound of formula I is formulated as immediate release film-coated tablet intended for oral administration. Tablets containing compound of formula I in this example are made in two strengths, about 5 mg and about 7 mg. The product containing compound of formula I is a white film-coated tablet encapsulated in a brownish red hard capsule. Other strengths, such as ˜2, ˜3, ˜4, ˜6, ˜8, ˜9, ˜10, ˜11, ˜12, ˜13, or ˜14 mg, may be prepared in the same manner.

### Composition

The compositions of the tablets 5 mg and 7 mg are given below in Table 1.

TABLE 1
Composition of tablets 5 mg and 7 mg

| | Quantity per Unit | | | | | Reference to |
|---|---|---|---|---|---|---|
| Name of Ingredient | 5 mg | | 7 mg | | Function | Standard¹ |
| DRUG SUBSTANCE | | | | | | |
| compound of formula I succinate | 6.665 | mg | 9.331 | mg | Active ingredient | In-house spec. |
| corresponding to compound of | 5 | mg | 7 | mg | | |
| formula I | | | | | | |
| EXCIPIENTS | | | | | | |
| Tablet core: | | | | | | |
| Calcium hydrogen | 37.990 | mg | 36.213 | mg | Filler | Ph. Eur. |
| phosphate, anhydrous | | | | | | |
| Maize starch | 18.995 | mg | 18.106 | mg | Filler | Ph. Eur. |
| Copovidone | 3.35 | mg | 3.35 | mg | Binder | Ph. Eur. |
| Water, purified² | q.s. | | q.s. | | Granulation liquid | Ph. Eur. |
| Cellulose, microcrystalline | 25 | mg | 25 | mg | Filler | Ph. Eur. |
| Croscarmellose sodium | 3 | mg | 3 | mg | Disintegrant | Ph. Eur. |
| Talc | 4 | mg | 4 | mg | Lubricant | Ph. Eur. |
| Magnesium stearate | 1 | mg | 1 | mg | Lubricant | Ph. Eur. |
| Weight of each tablet core | 100 | mg | 100 | mg | | |
| Film-coating: | | | | | | |
| Opadry Y-1-7000 white | | | | | | |
| consisting of: | | | | | | |
| Hypromellose (5 mPa · s.) | 1.563 | mg | 1.563 | mg | Film former | Ph. Eur. |
| Macrogol 400 | 0.156 | mg | 0.156 | mg | Plasticizer | Ph. Eur. |
| Titanium dioxide (E171) | 0.781 | mg | 0.781 | mg | Pigment | Ph. Eur. |
| Water, purified² | q.s. | | q.s. | | Solvent | Ph. Eur. |
| Weight of each film-coated tablet | 102.5 | mg | 102.5 | mg | | |

| | | | | |
|---|---|---|---|---|
| Magnesium stearate | q.s. | q.s. | Lubricant | Ph. Eur. |

¹The current pharmacopoeia is used
²Volatile material

The batch compositions for a representative batch size of 10,000 tablets are presented in Table 2.

TABLE 2
Batch composition for film-coated tablets (Batch size 10,000 tablets)

| | Strength | | | |
|---|---|---|---|---|
| | 5 mg | | 7 mg | |
| | | % w/w (per | | % w/w (per |
| Ingredients | Quantity (g) | tablet core) | Quantity (g) | tablet core) |
| Tablet core: | | | | |
| compound of formula I | 66.65 | 6.665 | 93.31 | 9.331 |
| succinate | | | | |
| Calcium hydrogen | 379.90 | 37.990 | 362.13 | 36.213 |
| phosphate, anhydrous | | | | |
| Maize starch | 189.95 | 18.995 | 181.06 | 18.106 |
| Copovidone | 33.5 | 3.35 | 33.5 | 3.35 |
| Water, purified¹ | q.s. | — | q.s. | — |
| Cellulose, microcrystalline | 250 | 25 | 250 | 25 |
| Croscarmellose sodium | 30 | 3 | 30 | 3 |
| Talc | 40 | 4 | 40 | 4 |
| Magnesium stearate | 10 | 1 | 10 | 1 |
| Weight of tablet core | 100 mg | | 100 mg | |
| Film coating: | | | | |
| Opadry Y-1-7000 white | 25 | 2.5 | 25 | 2.5 |
| Water, purified¹ | q.s. | — | q.s. | — |

| | | |
|---|---|---|
| Weight of film-coated tablet | 102.5 mg | 102.5 mg |

Description of Manufacturing Process and Process Controls

The method of granulation is a traditional wet granulation process using copovidone (Kollidone VA64) as a dry binder and water as granulation liquid. In the 10-litre PMA1 high shear mixer the process is as follows for a 2 kg batch:

Mix compound of formula I succinate (i.e., the succinate salt of a compound of formula I), anhydrous calcium hydrogen phosphate, maize starch and copovidone for 2 minutes at 500 rpm.

Add purified water to initiate agglomeration.

Granulate at 800 rpm for approximately 4 minutes, so a suitable granule size is achieved.

Sieve the Wet Granules.

Dry the granules in a tray dryer at 50° C., until the product has a relative humidity (RH) of 25-55%.

Sieve the Dried Granules.

Mix the granules with microcrystalline cellulose, croscarmellose sodium and talc in a mixer.

Add magnesium stearate to the mixer and mix.

Compress the granulate into tablets on a tablet compressing machine.

Film-coat the tablet cores in a film coater, using the process parameters given in Table 3.

TABLE 3
Equipment and process conditions for the coating process

| | | Spray | Inlet | Inlet | Outlet |
|---|---|---|---|---|---|
| | Load | rate | air flow | air temp. | air temp. |
| Equipment | (g) | (g/min) | (m³/h) | (° C.) | (° C.) |
| Compu Lab 15″ | 1360-1500 | 10 | 500 | 60 | 58 |

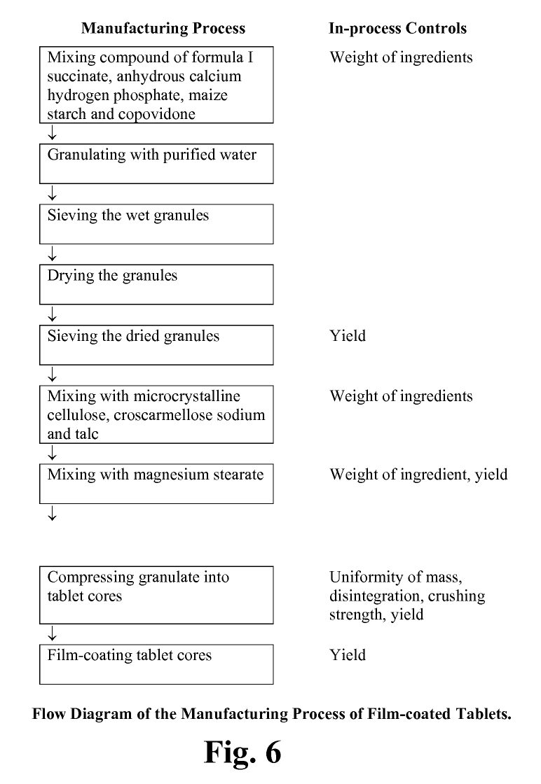

A flow diagram of the manufacturing process and process controls is shown in FIG. 6.

Unexpected Eeffects of Binder in the Tablet Formulation—Study I

In order to optimise the agglomeration process, two different tablet formulations was produced and their effect on the chemical stability of compound of formula I was evaluated.

The composition of these tablets is given in Table 4, and the manufacturing process, was similar to the one described above.

TABLE 4
Batch composition of film-coated tablets with 2 different binders
(Batch size 10,000 tablets)

Strength 2.5 mg

| | % w/w (per | % w/w (per |
|---|---|---|
| Ingredients | tablet core) | tablet core) |
| Tablet core: | | |
| compound of formula I | 2.67 | 2.67 |
| succinate | | |
| Calcium hydrogen | 40.66 | 40.66 |
| phosphate, anhydrous | | |
| Maize starch | 20.33 | 20.33 |
| Copovidone | 3.3 | 0.0 |
| Maltodextrin | 0.00 | 3.35 |
| Water, purified¹ | — | — |
| Cellulose, | 26.0 | 26.0 |
| microcrystalline | | |
| Croscarmellose | 3.0 | 3.0 |
| sodium | | |
| Talc | 3.0 | 3.0 |
| Magnesium stearate | 1.0 | 1.0 |

| | |
|---|---|
| Weight of tablet | 125 mg |
| core | |

The use of copovidone as binder leads to tablets with better pharmaceutical technical properties, e.g., the capability of producing harder tablets with low loss on friability without compromising the disintegration time, as demonstrated in Table 5:

TABLE 5
Comparison of pharmaceutical technical data for tablets containing compound
of formula I succinate with the composition given in Table 4

| Copovidone | | | Maltodextrin | | |
|---|---|---|---|---|---|
| Applied | | | Applied | | |
| compression | Friability | Disintegration | compression | Friability | |
| force (N) | (%, w/w) | time | force (N) | (%) | |
| 86 | 0.14 | 44 sec | 36 | 0.69 | 43 sec |
| 108 | 0.16 | 1 min 14 sec | 47 | 0.51 | 1 min 13 sec |
| 120 | 0.18 | 1 min 52 sec | 51 | 0.43 | 1 min 42 sec |
| 130 | 0.22 | 2 min 09 sec | 59 | 0.23 | 1 min 59 sec |

Furthermore, the difference in binder lead to surprising stability differences as demonstrated in Table 6.

TABLE 6
Decomposition of compound of formula I succinate, in
formulations where maltodextrin and copovidon are used
as binder, composition of tablets given in Table 4

| | Total decomposition (%) of compound of formula I | |
|---|---|---|
| Treatment | Copovidone | Maltodextrin |
| Initial analysis | <0.05 | <0.05 |
| After autoclavation | 0.91 | 1.1 |
| 80° C. for 48 hours | 0.99 | 2.0 |
| 80° C. for 120 hours | 1.4 | 3.7 |
| 40° C./75% RH | <0.05 | <0.05 |
| for 3 weeks | | |
| 60° C. for 3 weeks | 0.95 | 1.41 |

### Example 5

### Preparation of Immediate Release Film-Coated Tablet Intended for Oral Administration II

### Pharmaceutical Development

A study of the compatibility of the excipients and Compound I demonstrated that the components used in the tablet formulation were compatible with the compound. Based on this, a traditional wet granulation, tabletting and film-coating process was developed using standard methods and excipients.

Description of Drug product

Compound I is formulated as immediate release film-coated tablet intended for oral administration. Tablets containing compound of formula I in this example are made in two strengths, about 2.5 mg and about 5 mg. The product containing compound of formula I is a white film-coated tablet encapsulated in a brownish red hard capsule. Other strengths, such as about ˜2, ˜3, ˜4, ˜6, ˜7, ˜8, ˜9, ˜10, ˜11, ˜12, ˜13, or ˜14 mg, may be prepared in the same manner.

### Composition

The compositions of the tablets 2.5 mg and 5 mg are given below in Table 7.

TABLE 7
Composition of tablets 2.5 mg and 5 mg (calcium phosphate formulation)

| | Quantity per Unit | | | | | Reference to |
|---|---|---|---|---|---|---|
| Name of Ingredient | 2.5 mg | | 5 mg | | Function | Standard¹ |
| DRUG SUBSTANCE | | | | | | |
| Compound I, succinate | 3.333 | mg | 6.667 | mg | Active ingredient | In-house spec. |
| Corresponding to Compound I | 2.5 | mg | 5 | mg | | |
| EXCIPIENTS | | | | | | |
| Tablet core: | | | | | | |
| Calcium hydrogen | 40.000 | mg | 80.000 | mg | Filler | Ph. Eur. |
| phosphate, anhydrous | | | | | | |
| Maize starch | 20.000 | mg | 40.000 | mg | Filler | Ph. Eur. |
| Copovidone | 5.00 | mg | 10.00 | mg | Binder | Ph. Eur. |
| Water, purified² | q.s. | | q.s. | | Granulation liquid | Ph. Eur. |
| Cellulose, microcrystalline | 26.17 | mg | 52.34 | mg | Filler | Ph. Eur. |
| Croscarmellose sodium | 3 | mg | 6 | mg | Disintegrant | Ph. Eur. |
| Talc | 1.5 | mg | 3 | mg | Lubricant | Ph. Eur. |
| Magnesium stearate | 1 | mg | 2 | mg | Lubricant | Ph. Eur. |
| Weight of each tablet core | 100 | mg | 200 | mg | | |
| Film-coating: | | | | | | |
| Opadry Y-1-7000 white | | | | | | |
| consisting of: | | | | | | |
| Hypromellose (5 mPa · s.) | 1.563 | mg | 3.126 | mg | Film former | Ph. Eur. |
| Macrogol 400 | 0.156 | mg | 0.312 | mg | Plasticizer | Ph. Eur. |
| Titanium dioxide (E171) | 0.781 | mg | 1.562 | mg | Pigment | Ph. Eur. |
| Water, purified² | q.s. | | q.s. | | Solvent | Ph. Eur. |
| Weight of each film-coated tablet | 102.5 | mg | 205 | mg | | |

| | | | | |
|---|---|---|---|---|
| Magnesium stearate | q.s. | q.s. | Lubricant | Ph. Eur. |

¹The current pharmacopoeia is used
²Volatile material

The batch compositions for a representative batch size of 10,000 tablets are presented in Table 8.

TABLE 8
Batch composition for film-coated tablets (Batch size 10,000 tablets)

| | Strength | | | |
|---|---|---|---|---|
| | 2.5 mg | | 5 mg | |
| | | % w/w (per | | % w/w (per |
| Ingredients | Quantity (g) | tablet core) | Quantity (g) | tablet core) |
| Tablet core: | | | | |
| Compound of formula I | 33.33 | 3.333 | 66.67 | 3.333 |
| succinate | | | | |
| Calcium hydrogen | 400.00 | 40.000 | 800.00 | 40.000 |
| phosphate, anhydrous | | | | |
| Maize starch | 200.00 | 20.000 | 400.00 | 20.000 |
| Copovidone | 50.0 | 5.00 | 100.0 | 5.00 |
| Water, purified¹ | q.s. | — | q.s. | — |
| Cellulose, microcrystalline | 261.7 | 26.17 | 523.4 | 26.17 |
| Croscarmellose sodium | 30 | 3 | 60 | 3 |
| Talc | 15 | 1.5 | 30 | 1.5 |
| Magnesium stearate | 10 | 1 | 20 | 1 |
| Weight of tablet core | 100 mg | | 200 mg | |
| Film coating: | | | | |
| Opadry Y-1-7000 white | 25 | 2.5 | 50 | 2.5 |
| Water, purified¹ | q.s. | — | q.s. | — |

| | | |
|---|---|---|
| Weight of film-coated tablet | 102.5 mg | 205 mg |

Manufacturing process and process controls is as in Example 4.

A flow diagram of the manufacturing process and process controls is shown in FIG. 6.

Unexpected Effects of Binder in the Tablet Formulation—Study II

In order to optimise the agglomeration process, one tablet formulation (about 2.5 mg) for each binder was produced and the effect of binder on the chemical stability of Compound I was evaluated. The composition of these tablets is given in Table 9, and the manufacturing process, was similar to the one described above.

TABLE 9
Batch composition of film-coated tablets with 7 different binders
(Batch size 10,000 tablets)

| | Strength 2.5 mg | | | |
|---|---|---|---|---|
| | % w/w (per | % w/w (per | % w/w (per | % w/w (per |
| | tablet core) | tablet core) | tablet core) | tablet core) |
| | Formulation no.: | | | |
| Ingredients | 1 | 2 | 3 | 4 |
| Tablet core: | | | | |
| Compound of formula I | 3.33 | 3.33 | 3.33 | 3.33 |
| succinate | | | | |
| Calcium hydrogen | 40.66 | 40.66 | 40.66 | 40.66 |
| phosphate, anhydrous | | | | |
| Maize starch | 20.33 | 20.33 | 20.33 | 20.33 |
| Pregelatinized starch | 5.0 | 0.0 | 0.0 | 0.0 |
| Hypromellose | 0.0 | 5.0 | 0.0 | 0.0 |
| Povidone | 0.0 | 0.0 | 5.0 | 0.0 |
| Methylcellulose | 0.0 | 0.0 | 0.0 | 5.0 |
| Water, purified¹ | — | — | — | — |
| Cellulose, microcrystalline | 25.2 | 25.2 | 25.2 | 25.2 |
| Croscarmellose sodium | 3.0 | 3.0 | 3.0 | 3.0 |
| Talc | 1.5 | 1.5 | 1.5 | 1.5 |
| Magnesium stearate | 1.0 | 1.0 | 1.0 | 1.0 |

| | |
|---|---|
| Weight of tablet core | 100 mg |

| | Strength 2.5 mg | | |
|---|---|---|---|
| | % w/w (per | % w/w (per | % w/w (per |
| | tablet core) | tablet core) | tablet core) |
| | Formulation no.: | | |
| Ingredients | 5 | 6 | 7 |
| Tablet core: | | | |
| compound of formula I succinate | 3.33 | 3.33 | 2.67 |
| Calcium hydrogen | 40.66 | 40.00 | 40.66 |
| phosphate, anhydrous | | | |
| Maize starch | 20.33 | 20.00 | 20.33 |
| Sucrose | 5.0 | 0.0 | 0.0 |
| Copovidone | 0.0 | 5.0 | 0.0 |
| Maltodextrin | 0.0 | 0.0 | 3.35 |
| Water, purified¹ | — | — | — |
| Cellulose, microcrystalline | 25.2 | 26.2 | 26.0 |
| Croscarmellose sodium | 3.0 | 3.0 | 3.0 |
| Talc | 1.5 | 1.5 | 3.0 |
| Magnesium stearate | 1.0 | 1.0 | 1.0 |
| Weight of tablet core | 100 mg | 100 mg | 125 mg |

The use of copovidone as binder (Formulation No. 6) leads to tablets with good pharmaceutical technical properties, e.g., a relative long disintegration time permitting the tablets to be swallowed as whole tablets (as demonstrated in Table 10) and acceptable stability data (as demonstrated in Table 11 and Table 12):

TABLE 10
Comparison of pharmaceutical technical data for
tablets containing compound of formula I succinate
with the composition given in Table 9

| Pharm. | Weight of the | | Friability | Disintegration |
|---|---|---|---|---|
| Technical data | tablet core | Hardness | (16 min) | (sec.) |
| Form. 1 | 100 mg | 46 N | 0.5% | 11 |
| Form. 2 | 100 mg | 50 N | 0.6% | 22 |
| Form. 3 | 100 mg | 48 N | 0.5% | 35 |
| Form. 4 | 100 mg | 53 N | — | 39 |
| Form. 5 | 100 mg | 63 N | — | 45 |
| Form. 6 | 100 mg | 37 N | 0.5% | 112 |
| Form. 7 | 125 mg | 36 N | 0.7% | 43 |

Some differences in the stability of the products containing different binders can be seen in Tables 11 and 12 (next page).

TABLE 11
Decomposition of compound of formulations 1 to 6 - different
binders are used, composition of tablets given in Table 9

| | Total decomposition (%) of API | | | | | |
|---|---|---|---|---|---|---|
| Treatment | Form. 1 | Form. 2 | Form. 3 | Form. 4 | Form. 5 | Form. 6 |
| Initial analysis | ND | ND | ND | ND | ND | ND |
| Autoclavation | 0.43 | 0.44 | 0.94 | 0.51 | 0.99 | 0.53 |
| 80° C. for 48 | 2.6 | 3.2 | 9.7 | 3.4 | 1.4 | 5.4 |
| hours (open) | | | | | | |
| 80° C. for 48 | 5.3 | 1.7 | 5.2 | 2.0 | 1.9 | 5.9 |
| hours (closed) | | | | | | |
| 80° C. for 144 | 5.0 | 6.8 | 20.0 | 6.6 | 2.6 | 12.7 |
| hours (open) | | | | | | |
| 80° C. for 144 | 2.7 | 4.5 | 9.0 | 3.8 | 5.1 | 2.9 |
| hours (closed) | | | | | | |
| 40° C./75% | 0.17 | 0.18 | 0.25 | 0.25 | 0.17 | 0.32 |
| RH for 1 week | | | | | | |
| 40° C./75% | 0.18 | 0.28 | 0.34 | 0.30 | 0.25 | 0.31 |
| RH for 3 weeks | | | | | | |
| 40° C./75% | 0.25 | 0.30 | 0.43 | 0.35 | 0.35 | 0.41 |
| RH for 6 weeks | | | | | | |
| 40° C./75% | 0.30 | 0.36 | 0.70 | 0.38 | 0.54 | 0.66 |
| RH for 10 | | | | | | |
| weeks | | | | | | |
| 40° C./75% | 0.33 | 0.36 | 0.80 | 0.41 | 0.60 | 0.75 |
| RH for 12 | | | | | | |
| weeks | | | | | | |
| 60° C. for | 0.59 | 0.55 | 1.1 | 0.61 | 0.28 | 0.69 |
| 1 week | | | | | | |
| 60° C. for | 1.6 | 1.5 | 3.5 | 1.6 | 0.48 | 1.8 |
| 3 weeks | | | | | | |
| 60° C. for | 2.4 | 2.4 | 6.2 | 2.5 | 0.88 | 2.9 |
| 6 weeks | | | | | | |
| 60° C. for | 3.5 | 3.6 | 9.6 | 3.9 | 1.2 | 4.6 |
| 10 weeks | | | | | | |
| 60° C. for | 3.7 | 3.8 | 10.3 | 4.2 | 1.4 | 5.0 |
| 12 weeks | | | | | | |

ND = Not detected

TABLE 12
Decomposition of compound of formulation 7 -
formulation where maltodextrin is used as binder,
composition of tablets given in Table 9

| Treatment | |
|---|---|
| Binder | Maltodextrin (form. 7) |
| Initial analysis | <0.05 |
| After autoclavation | 1.1 |
| 80° C. for 48 hours | 2.0 |
| 80° C. for 120 hours | 3.7 |
| 40° C./75% RH | <0.05 |
| for 3 weeks | |
| 60° C. for 3 weeks | 1.41 |

### Example 6

### Preparation of Immediate Release Film-Coated Tablet Intended for Oral Administration III

### Pharmaceutical Development

A study of the compatibility of the excipients and Compound I demonstrated that the components used in the tablet formulation were compatible with the compound. Based on this, a traditional wet granulation, tabletting and film-coating process was developed using standard methods and excipients.

### Description of Drug Product

Compound I is formulated as immediate release film-coated tablet intended for oral administration. Tablets containing compound of formula I in this example are made in two strengths, about 2.5 mg and about 5 mg. The product containing compound of formula I is a white film-coated tablet encapsulated in a brownish red hard capsule. Other strengths, such as ˜2, ˜3, ˜4, ˜6, ˜7, ˜8, ˜9, ˜10, ˜11, ˜12, ˜13, or ˜14 mg, may be prepared in the same manner.

### Composition

The compositions of the tablets 2.5 mg and 5 mg are given below in Table 13 and Table 14:

TABLE 13
Composition of tablets 2.5 mg and 5 mg (calcium phosphate formulation)

| | Quantity per Unit | | | | | Reference to |
|---|---|---|---|---|---|---|
| Name of Ingredient | 2.5 mg | | 5 mg | | Function | Standard¹ |
| DRUG SUBSTANCE | | | | | | |
| Compound I, succinate | 3.333 | mg | 6.667 | mg | Active ingredient | In-house spec. |
| Corresponding to Compound I | 2.5 | mg | 5 | mg | | |
| EXCIPIENTS | | | | | | |
| Tablet core: | | | | | | |
| Calcium hydrogen | 40.000 | mg | 40.000 | mg | Filler | Ph. Eur. |
| phosphate, anhydrous | | | | | | |
| Maize starch | 20.000 | mg | 20.000 | mg | Filler | Ph. Eur. |
| Copovidone | 5.00 | mg | 5.00 | mg | Binder | Ph. Eur. |
| Water, purified² | q.s. | | q.s. | | Granulation liquid | Ph. Eur. |
| Cellulose, microcrystalline | 26.17 | mg | 22.83 | mg | Filler | Ph. Eur. |
| Croscarmellose sodium | 3 | mg | 3 | mg | Disintegrant | Ph. Eur. |
| Talc | 1.5 | mg | 1.5 | mg | Lubricant | Ph. Eur. |
| Magnesium stearate | 1 | mg | 1 | mg | Lubricant | Ph. Eur. |
| Weight of each tablet core | 100 | mg | 100 | mg | | |
| Film-coating: | | | | | | |
| Opadry Y-1-7000 white | | | | | | |
| consisting of: | | | | | | |
| Hypromellose (5 mPa · s.) | 1.563 | mg | 1.563 | mg | Film former | Ph. Eur. |
| Macrogol 400 | 0.156 | mg | 0.156 | mg | Plasticizer | Ph. Eur. |
| Titanium dioxide (E171) | 0.781 | mg | 0.781 | mg | Pigment | Ph. Eur. |
| Water, purified² | q.s. | | q.s. | | Solvent | Ph. Eur. |
| Weight of each film-coated tablet | 102.5 | mg | 102.5 | mg | | |

| | | | | |
|---|---|---|---|---|
| Magnesium stearate | q.s. | q.s. | Lubricant | Ph. Eur. |

¹The current pharmacopoeia is used
²Volatile material

TABLE 14
Composition of tablets 2.5 mg and 5 mg (lactose formulation)

| | Quantity per Unit | | | | | Reference to |
|---|---|---|---|---|---|---|
| Name of Ingredient | 2.5 mg | | 5 mg | | Function | Standard¹ |
| DRUG SUBSTANCE | | | | | | |
| Compound I, succinate | 3.333 | mg | 6.667 | mg | Active ingredient | In-house spec. |
| Corresponding to Compound I | 2.5 | mg | 5 | mg | | |
| EXCIPIENTS | | | | | | |
| Tablet core: | | | | | | |
| Lactose | 39.330 | mg | 39.330 | mg | Filler | Ph. Eur. |
| Maize starch | 15.000 | mg | 15.000 | mg | Filler | Ph. Eur. |
| Copovidone | 3.35 | mg | 3.35 | mg | Binder | Ph. Eur. |
| Water, purified² | q.s. | | q.s. | | Granulation liquid | Ph. Eur. |
| Cellulose, microcrystalline | 34.99 | mg | 31.65 | mg | Filler | Ph. Eur. |
| Croscarmellose sodium | 3 | mg | 3 | mg | Disintegrant | Ph. Eur. |
| Magnesium stearate | 1 | mg | 1 | mg | Lubricant | Ph. Eur. |
| Weight of each tablet core | 100 | mg | 100 | mg | | |
| Film-coating: | | | | | | |
| Opadry Y-1-7000 white | | | | | | |
| consisting of: | | | | | | |
| Hypromellose (5 mPa · s.) | 1.563 | mg | 1.563 | mg | Film former | Ph. Eur. |
| Macrogol 400 | 0.156 | mg | 0.156 | mg | Plasticizer | Ph. Eur. |
| Titanium dioxide (E171) | 0.781 | mg | 0.781 | mg | Pigment | Ph. Eur. |
| Water, purified² | q.s. | | q.s. | | Solvent | Ph. Eur. |
| Weight of each film-coated tablet | 102.5 | mg | 102.5 | mg | | |

| | | | | |
|---|---|---|---|---|
| Magnesium stearate | q.s. | q.s. | Lubricant | Ph. Eur. |

¹The current pharmacopoeia is used
²Volatile material

Each reference cited in the present application, including literature references, books, patents and patent applications, is incorporated herein by reference in its entirety.

What is claimed is:

1. A method of improving cognitive functioning, comprising administering an effective amount of trans-4-((1R,3S)-6-chloro-3-phenylindan-1-yl)-1,2,2-trimethylpiperazine or a pharmaceutically acceptable salt thereof, to a patient in need thereof.

2. The method according to claim 1, wherein the patient suffers from cognitive dysfunction.

3. A method of treating cognitive dysfunction in connection with a disease, comprising administering an effective amount of trans-4-((1R,3S)-6-chloro-3-phenylindan-1-yl)-1,2,2-trimethylpiperazine or a pharmaceutically acceptable salt thereof to a patient in need thereof, wherein the disease is selected from the group consisting of schizophrenia, a disease involving a psychotic symptom, schizophreniform disorder, schizoaffective disorder, delusional disorder, brief psychotic disorder, shared psychotic disorder, substance-induced psychotic disorder, an affective disorder, Parkinson's disease, a disease involving a sleep disturbance, neuroleptic-induced parkinsonism, and an abuse disorder.

4. The method according to claim 3, wherein the abuse disorder is selected from the group consisting of cocaine abuse, nicotine abuse, and alcohol abuse.

5. The method according to claim 3, wherein the affective disorder is selected from the group consisting of depression, bipolar disorder and mania.

6. The method according to claim 3, wherein the disease is schizophrenia.

7. The method according to claim 6, wherein the method further comprises reducing a cognitive symptom in a schizophrenic patient.

8-22. (canceled)

23. A method of treating cognitive impairment associated with schizophrenia (CIAS), comprising administering an effective amount of trans-4-((1R,3S)-6-chloro-3-phenylindan-1-yl)-1,2,2-trimethylpiperazine or a pharmaceutically acceptable salt thereof to a patient in need thereof.

24-26. (canceled)

27. A method of treating first-episode schizophrenia, comprising administering an effective amount of trans-4-((1R,3S)-6-chloro-3-phenylindan-1-yl)-1,2,2-trimethylpiperazine or a pharmaceutically acceptable salt thereof to a patient in need thereof.

28-38. (canceled)
